(19) United States
(12) Patent Application Publication
(10) Pub. No.: US 2011/0130296 A1
(43) Pub. Date: Jun. 2, 2011

(54) MULTI-GENE CLASSIFIERS AND PROGNOSTIC INDICATORS FOR CANCERS

(75) Inventors: Christopher Benz, Novato, CA (US); Laura Esserman, San Francisco, CA (US); Frederic Waldman, San Francisco, CA (US); Christina Yau, Novato, CA (US)

(73) Assignees: The Regents of the University of California; The buck Institute

(21) Appl. No.: 12/922,651

(22) PCT Filed: Mar. 10, 2009
(86) PCT No.: PCT/US09/36672

§ 371 Date: Jan. 3, 2011

Related U.S. Application Data
(60) Provisional application No. 61/036,861, filed on Mar. 14, 2008.

(51) Int. Cl. C40B 30/00; C40B 40/06; C12Q 1/68

(52) U.S. Cl. 506/7; 506/16; 435/6.12; 435/6.14

(57) ABSTRACT

The present invention relates to the identification of marker genes useful in the diagnosis and prognosis of clinically problematic subsets of primary breast cancers. More specifically, the invention relates to the identification of two sets of marker genes that are differentially expressed in and useful for the diagnosis and prognosis of subsets of hormone receptor-negative (HRneg; i.e., ER and PR negative) and triple-negative (Tneg; i.e., ER, PR and HER2 negative) primary breast cancers at highest risk for early metastatic relapse. The invention further provides methods for determining the best course of treatment for patients having one of these clinically problematic subsets of primary breast cancers. The invention also provides methods for identifying compounds that prevent or treat a subtype of breast cancer based on their ability to modulate the activity or expression level of one or more marker genes identified herein.

### CROSS-REFERENCES TO RELATED APPLICATIONS

This application claims priority to U.S. Provisional Appl. No. 61/036,861, filed Mar. 14, 2008, the disclosure of which is incorporated by reference in its entirety.

### STATEMENT AS TO RIGHTS TO INVENTIONS MADE UNDER FEDERALLY SPONSORED RESEARCH OR DEVELOPMENT

This invention was made with Government support under NCI Grant No. P50-CA58207. The Government has certain rights in this invention.

### BACKGROUND OF THE INVENTION

Breast cancer is the second most common cancer in women, after skin cancer, and the second leading cause of cancer-related death in women, after lung cancer. The American Cancer Society estimates that one in every eight women will have invasive breast cancer some time during her life. Further, they estimate that one in every thirty-five women will die because of it. Breast cancer is a malignant tumor that initiates from cells of the breast. Early detection of and proper diagnosis of the specific subtype of breast cancer can significantly increase the survival rate of an individual with breast cancer. The ability to treat and potentially cure early forms of breast cancer underscores the need for more accurate diagnostic methods for both the early detection of this disease and for better markers to serve as prognosticators of disease subtype and progression to afford better informed medical treatment strategies.

Marker-based approaches to tumor identification and characterization have shown early promise for improved diagnostic and prognostic reliability. Histopathological examinations are generally relied on for the diagnosis of breast cancer and typically provide information about the prognosis and selection of treatment regimens. Prognosis may also be established based upon clinical parameters such as tumor size, tumor grade, the age of the patient, and lymph node metastasis. However, these types of analysis commonly fail to identify and accurately prognosticate the fate of clinically problematic subtypes of breast cancer, such as hormone receptor-negative (HRneg; i.e., estrogen receptor (ER) and progesterone receptor (PR) negative) and triple-negative (Tneg; i.e., ER, PR and HER2 negative).

In clinical practice, accurate diagnosis of these problematic subtypes of breast cancer is of critical important due to the fact that treatment options, prognosis, and the likelihood of therapeutic response all vary broadly dependent upon on the breast cancer subtype. Accurate diagnosis and prognosis allows the practitioner to tailor the treatment plan for maximal efficacy. Furthermore, accurate prediction of poor prognosis allows for the stratification of patients who may benefit the most from clinical trials and experimental therapy.

While the mechanism of tumorigenesis for most breast carcinomas is largely unknown, there are genetic factors that can predispose some women to developing breast cancer (Miki et al., Science, 266:66 71 (1994)). For example, BRCA1 and BRCA2 are genetic factors which can contribute to familial breast cancer. Germ-line mutations within these two loci are associated with a greater than 50% lifetime risk of breast and/or ovarian cancer (Casey, Curr. Opin. Oncol. 9:88 93 (1997); Marcus et al., Cancer 77:697 709 (1996)). However, only about 5% to 10% of breast cancers are associated with breast cancer susceptibility genes, BRCA1 and BRCA2. The cumulative lifetime risk of breast cancer for women who carry the mutant BRCA1 is predicted to be greater than 90%, while the cumulative lifetime risk for the non-carrier majority is estimated to be approximately 10%.

Other genes have been linked to breast cancer, for example c-erb-2 (HER2) and p53 (Beenken et al., Ann. Surg. 233(5):630 638 (2001). Overexpression of c-erb-2 (HER2) and p53 have been correlated with poor prognosis (Rudolph et al., Hum. Pathol. 32(3):311 319 (2001), as has been aberrant expression products of mdm2 (Lukas et al., Cancer Res. 61(7):3212 3219 (2001) and cyclin1 and p27 (Porter & Roberts, International Publication WO98/33450, published Aug. 6, 1998).

The recent advent of gene array profiling has improved the diagnostic and prognostic powers of cancer linked markers. For example, Perou et al. showed that there are several subgroups of breast cancer patients based on unsupervised cluster analysis of cDNA microarrays (Perou et al., Nature 406(6797):747 752 (2000)). Sorlie et al., (PNAS, 98(19):10869 10874 (2001)) demonstrated that these subgroups differ with respect to outcome of disease in patients with locally advanced breast cancer. This technology has also been used to identify diagnostic categories, e.g., BRCA1 and BRCA2 related cancers (Hedenfalk et al., N. Engl. J. Med. 344(8):539 548 (2001). However, no validated prognostic gene signatures have been identified for the clinically problematic subsets of HRneg and Tneg primary breast cancers at highest risk for early metastatic relapse. This is especially true when these subsets are considered independent of the larger and more well-defined subset of HRpos breast cancers. The current invention solves this problem through the identification of several prognostic gene marker sets for the breast cancer subtypes HRneg and Tneg.

### BRIEF SUMMARY OF THE INVENTION

Generally, the methods of this invention find particular use in diagnosing or providing a prognosis for hormone receptor-negative (HRneg; i.e., ER and PR negative) and triple-negative (Tneg; i.e., ER, PR and HER2 negative) primary breast cancers by detecting the expression levels of gene markers, which are differentially expressed (down or upregulated) in breast cancer cells of these specific subtypes and correlate with disease progression. These markers can thus be used diagnostically to distinguish the HRneg and Tneg breast cancer subtypes from other, generally less clinically problematic subtypes. They can also be used prognostically for patient risk assessment, to determine the probability of overall survival, sentinel lymph node (SLN) status, relapse free survival, and/or disease specific survival.

By categorizing HRneg and Tneg breast cancer cases at the time of diagnosis according to higher or lower risk of developing metastatic recurrence, these markers are able to predict which cases need little or no systemic adjuvant/neoadjuvant chemotherapy from those needing very aggressive adjuvant/neoadjuvant chemotherapy, which is currently recommended for almost all newly diagnosed HRneg and Tneg breast cancer cases given the absence of such prognostic markers.

The markers can be used alone or in combination for risk assessment. These markers can also be used individually or in combination to predict individual HRneg or Tneg cases that will be more or less likely to benefit from treatment with specific chemotherapeutics (as single drugs or in drug combinations). They can also be used to identify tumorigenic pathways in HRneg and Tneg breast (or other) cancers for the design and development of novel targeted agents to treat these cancers, and subsequently serve as predictive markers for responsiveness to these novel therapies.

Accordingly, the invention includes methods of providing a prognosis for an individual with a Hormone Receptor negative (HRneg) or Triple negative (Tneg) breast cancer subtype, said method comprising: (i) determining the gene expression profile of a breast cancer subtype tumor cell from the individual with respect to a marker set useful for the prognosis of a HRneg or Tneg breast cancer subtype; and (ii) classifying said gene expression profile as indicating a high or low risk of metastatic relapse independent of therapy, wherein said marker set comprises at least one gene selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN, MCM6, ATG5, COL2A1, FKBP10, NPM1, CASPSAP2, CEACAM7, FBLX4, NPAS3, and SCGB2A2, thereby providing a prognosis for an individual with a HRneg or Tneg breast cancer subtype. In some embodiments, the marker set comprises at least 2, 3, 4, 5, 6, 7, 8, 9, 10, 11, 12, 13, 14, 15, 16, 17, 18, 19, 20, 21, 22, 23, or 24 of the marker genes in any combination.

In some embodiments, the marker set comprises at least one of the genes selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, and MATN. In some embodiments, the marker set comprises CXCL13. In some embodiments, the marker set comprises CLIC5 and CXCL13. In some embodiments, the marker set comprises CLIC5, CXCL13, PRTN3, FLJ46061/RPS28, SSX3, ABO, and RGS4. In some embodiments, the marker set comprises: CXCL13, HAPLN1, FLJ46061///RPS28, RGS4, SSX3, RFXDC2, EXOC7, CLIC5, ZNF3, PRRG3, ABO, PRTN3, HRBL, MATN

In some embodiments, the individual is HRneg. In some embodiments, the marker set is selected from the group consisting of: HRneg S1, HRneg S2, HRneg HP, and HRTCS. In some embodiments, the individual is Tneg. In some embodiments, the marker set is selected from the group consisting of: Tneg S1, Tneg S2, Tneg HP, and HRTCS.

In some embodiments, the expression profile is determined by RT-PCR. In some embodiments, the expression profile is determined by microarray In some embodiments, the expression profile is determined by immunoaffinity methods, such as a microarray or immunofluorescence.

In some embodiments, the methods of providing a prognosis further comprise the step of adjusting the therapy of the individual based on the prognosis. In some embodiments, the prognosis is a high risk of metastatic relapse independent of therapy, and the therapy is adjusted to be more aggressive, e.g., increasing the dose or frequency of chemotherapy or increasing the frequency of medical monitoring. In some embodiments, the prognosis is a low risk of metastatic relapse independent of therapy, and the therapy is adjusted to be less aggressive, e.g., reducing the dose or frequency of chemotherapy or reducing the frequency of medical monitoring.

The invention also provides methods of assigning treatment to an individual having an HRneg or Tneg breast cancer subtype, said method comprising: (i) providing a prognosis for the individual as described above; and (ii) assigning a treatment to the individual based on the prognosis provided in step (i).

The invention provides microarrays for determining the gene expression profile of a Hormone Receptor negative (HRneg) or Triple negative (Tneg) breast cancer subtype cell. Such microarrays comprise at least two oligonucleotide probes complimentary to genes selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN, MCM6, ATG5, COL2A1, FKBP10, NPM1, CASPSAP2, CEACAM7, FBLX4, NPAS3, and SCGB2A2. In some embodiments, the microarray comprises at least 3, 4, 5, 6, 7, 8, 9, 10, 11, 12, 13, 14, 15, 16, 17, 18, 19, 20, 21, 22, 23, or 24 of the recited probes in any combination.

In some embodiments, the microarray comprises at least two oligonucleotide probes complimentary to genes selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, and MATN. In some embodiments, the microarray comprises oligonucleotide probes complimentary to CXCL13. In some embodiments, the microarray comprises an oligonucleotide probe complimentary to CLIC5 and CXCL13. In some embodiments, the microarray comprises oligonucleotide probes complimentary to: CLIC5, CXCL13, PRTN3, FLJ46061/RPS28, SSX3, ABO, and RGS4. In some embodiments, the microarray comprises oligonucleotide probes complimentary to: CXCL13, HAPLN1, FLJ46061///RPS28, RGS4, SSX3, RFXDC2, EXOC7, CLIC5, ZNF3, PRRG3, ABO, PRTN3, HRBL, and MATN.

In some embodiments, the invention provides methods of identifying an agent useful for treatment of a Hormone Receptor negative (HRneg) or Triple negative (Tneg) breast cancer subtype, said method comprising: (i) detecting whether a breast cancer cell is HRneg or Tneg; (ii) contacting a HRneg or Tneg breast cancer cell detected in step (i) with a test agent; (iii) determining the level of expression of at least one marker gene selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN, MCM6, ATG5, COL2A1, FKBP10, NPM1, CASPSAP2, CEACAM7, FBLX4, NPAS3, and SCGB2A2 in the cell contacted in step (ii), wherein a difference between the level of expression between the cell contacted in step (ii) and an untreated control cell indicates the presence of an agent useful for treatment of an HRneg or Tneg breast cancer subtype. In some embodiments, the determining step comprises RT-PCR. In some embodiments, the determining step comprises microarray analysis. In some embodiments, the agent increases expression of the marker gene, e.g., where the marker is PRTN3, ABO, EXOC7, RFXDC2, PRRG3, CXCL13, CLIC5, FLJ46061///RPS28, HRBL, SSX3, ZNF3, or MATN.

In some embodiments, the untreated control is the breast cancer cell detected in step (i) prior to contacting with the test agent. In some embodiments, the untreated control is a breast cancer cell of the same subtype as the breast cancer cell detected in step (i). In some embodiments, the breast cancer subtype is HRneg and the marker gene is selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN, MCM6, ATG5, COL2A1, FKBP10, NPM1, CASPSAP2, and CEACAM7. In some embodiments, the breast cancer subtype is Tneg and the marker gene is selected from the group consisting of: FLJ46061///RPS28, CXCL13, HRBL, CLIC5, ZNF3, SSX3, MATN, FBLX4, NPAS3, and SCGB2A2.

In some embodiments, the invention provides methods of identifying an agent useful for treatment of a Hormone Receptor negative (HRneg) or Triple negative (Tneg) breast cancer subtype, said method comprising: (i) detecting whether a breast cancer cell is HRneg or Tneg; (ii) contacting a HRneg or Tneg breast cancer cell detected in step (i) with a test agent; (iii) determining the level of activity of at least one marker gene selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN, MCM6, ATG5, COL2A1, FKBP10, NPM1, CASPSAP2, CEACAM7, FBLX4, NPAS3, and SCGB2A2 in the cell contacted in step (ii), wherein a difference between the level of activity between the cell contacted in step (ii) and an untreated control cell indicates the presence of an agent useful for treatment of an HRneg or Tneg breast cancer subtype.

Diagnostic and prognostic kits comprising one or more markers of the invention are provided. Also provided by the invention are methods for identifying compounds that are able to prevent or treat breast cancer progression by modulating the markers found in any one of the identified gene subsets.

The invention also provides therapeutic methods, wherein a HRneg or Tneg breast cancer subtype is treated with a modulator of one of the marker genes described herein. In some embodiments, the modulator is an inhibitory polynucleotide that specifically binds to and inhibits expression of a marker of the invention, e.g., MCM6, ATG5, RGS4, HAPLN1, COL2A1, FKBP10, NPM1, or CASP8AP2. In some embodiments, the modulator is a coding sequence, e.g., to increase expression of PRTN3, ABO, EXOC7, RFXDC2, PRRG3, CXCL13, CLIC5, FLJ46061///RPS28, HRBL, SSX3, ZNF3, or MATN.

### BRIEF DESCRIPTION OF THE DRAWINGS

FIG. 1: Kaplan Meier analysis of dichotomized HRneg dataset based on individual probe expression.

FIG. 2: Kaplan Meier analysis of dichotomized Tneg dataset based on individual probe expression.

FIG. 3: Kaplan Meier analysis of dichotomized HRneg dataset based on summation index.

FIG. 4: Kaplan Meier analysis of dichotomized Tneg dataset based on summation index.

FIG. 5: Kaplan Meier analysis of dichotomized highest priority HRneg dataset based on summation index.

FIG. 6: Kaplan Meier analysis of dichotomized highest priority Tneg dataset based on summation index.

FIG. 7: Kaplan Meier analysis of dichotomized six gene Agilent HRneg dataset based on summation index.

FIG. 8: Kaplan Meier analysis of dichotomized 11 HRneg Gene Finalists based on individual probe expression.

FIG. 9: Kaplan Meier analysis of dichotomized 7 Tneg Gene Finalists based on individual probe expression.

FIG. 10: Kaplan Meier analysis of dichotomized 11 HRneg Gene Finalists (left panel) and 7 Tneg Gene Finalists (right panel) based on summation indices.

FIG. 11: Kaplan Meier analysis of dichotomized 14 gene panel based on 199 HRneg summation index (left panel) and 154 Tneg summation index (right panel).

FIG. 12: Comparison of prognostic value of 14 gene panel to five other breast cancer gene signatures in combined HRneg and Tneg samples. Kaplan Meier analysis of the present 14 gene panel summation index illustrates the strong predictive value compared to the 21 Gene Recurrence Signature, p53 Signature, 70 Gene Signature, Genomic Grade Index, and 7 Gene Immune Response Signature. COX analysis is shown in the bottom left.

### DETAILED DESCRIPTION OF THE INVENTION

While prognostic breast cancer gene expression profiles have recently been introduced into the clinic, to date there have been no validated prognostic gene signatures identified for the clinically problematic subsets of hormone receptor-negative (HRneg; i.e., ER and PR negative) and triple-negative (Tneg; i.e., ER, PR and HER2 negative) primary breast cancers at highest risk for early metastatic relapse. Despite their molecular and clinical heterogeneity, virtually all newly diagnosed HRneg and Tneg breast cancers are treated with standard adjuvant combination chemotherapy. The present invention develops multi-gene classifiers and outcome predictors to improve the clinical management of newly diagnosed, node-negative HRneg and Tneg breast cancer patients by identifying those at highest and lowest risk for metastatic relapse independent of therapy.

Other gene expression signatures seek to assign relapse risk to a given subset of breast cancers using microarray analysis or RT-PCR analysis on a full spectrum of breast cancer subtypes. For example, the PAM-50 classifier, aimed at predicting relapse in basal-like breast cancers, was recently developed from the earlier “Intrinsic Gene Signature” (Parker et al. (February 2009) J. Clin. Oncol.; Perreard et al. (2006) Breast Can. Res. 8:R23). Basal-like breast cancers are a subset of Tneg breast cancers which are a subset of HRneg breast cancers. The recently described PAM-50 signature depends on gene expression patterns that differentiate basal-like from other subtypes (Luminal A, Luminal B, HER2 enriched, normal-like, claudin). In contrast, our HRneg and Tneg signatures were derived using only HRneg or Tneg breast cancers selected clinically, and therefore can prognostically classify these subsets, not in relation to other breast cancer subsets, but in relation to the diversity within their own HRneg or Tneg subset. Therefore, there is an important distinction between how the present signatures perform based on how they were derived. The present signatures are also useful for distinct purposes in the clinic compared to breast cancer classifying signatures derived in a different manner.

A discovery/training set of 135 untreated, node-negative (N0), ER-negative primary breast cancers was identified from published studies which used the Affymetrix U133A microarray platform (Wang et al. (2005) Lancet 365:671-79, GSE2034; Minn et al. (2007) Proc. Natl. Acad. Sci. USA 104:6740-45, GSE5327). A subset of 108 cases was identified as Tneg based on the bimodal distribution of ERBB2 mRNA transcript levels.

Candidate probes/genes associated with metastasis-free survival from the discovery/training sets were subsequently assigned into hierarchical prioritizations based on their biostatistical evaluation against another untreated N0 dataset of 64 HRneg and 46 Tneg cases similarly analyzed using the Affymetrix platform (TRANSBIG; Desmedt et al., GSE7390). High priority candidates were further validated against 37 HRneg cases from Netherlands Cancer Institute (NKI) analyzed by a different microarray platform (Agilent) (van de Vijver et al. (2002) N Engl J Med 347:1999-2009). Multiple analytic approaches were used to prioritize candidate genes to create a flexible list for application to the Guy's set of tumors.

Using two different statistical methods (PAM and iterative sampling) and multivariate Cox modeling to ascertain the consistency of a biomarker's correlation with outcome, 18 genes were identified as HRneg prognostic candidates (HRneg S1), 10 genes were identified as Tneg prognostic candidates (Tneg S1), and 4 genes were common to both prognostic groups (Combined HRneg/Tneg Subset, or HRTCS) (see Table 1). When used in a summation index, these candidates were better able to predict metastasis-free survival than as single gene predictors. Following univariate Cox analysis against the TRANSBIG dataset, 11/18 HRneg (HRneg S2) and 6/10 Tneg candidates (Tneg S2) were assigned higher priority. Following multivariate Cox modeling, 8/18 HRneg candidates (HRneg Highest Priority, or HRneg SHP) and 5/10 Tneg candidates (Tneg Highest Priority, or Tneg SHP) were assigned highest priority (see Table 1). Of the 11 higher priority HRneg candidates, only 6 (MATN1, ABO, RGS4, PRTN3, CLIC5, RPS28) were available on the Agilent platform. These 6 markers, however, showed significant prognostic value as a summation index.

TABLE 1
Summary of genes identified as HRneg and/or Tneg prognostic

| | HRneg | HRneg | HRneg | Tneg | Tneg | Tneg | |
|---|---|---|---|---|---|---|---|
| Gene | S1 | S2 | HP | S1 | S2 | HP | HRTCS |
| MCM6 | + | | | | | | |
| ATG5 | + | | | | | | |
| RGS4 | + | + | | | | | |
| HAPLN1 | + | + | + | | | | |
| COL2A1 | + | | | | | | |
| FKBP10 | + | | | | | | |
| NPM1 | + | | | | | | |
| CASPSAP2 | + | | | | | | |
| CXCL13 | + | + | + | + | + | + | + |
| CEACAM7 | + | | | | | | |
| MATN1 | + | + | | + | + | + | + |
| PRTN3 | + | + | + | | | | |
| FLJ46061/ | + | + | + | + | | | + |
| RPS28 | | | | | | | |
| EXOC7 | + | + | + | | | | |
| ABO | + | + | | | | | |
| CLIC5 | + | + | + | + | + | + | + |
| RFXDC2 | + | + | + | | | | |
| PRRG3 | + | + | + | | | | |
| FBLX4 | | | | + | | | |
| HRBL | | | | + | + | | |
| ZNF3 | | | | + | + | + | |
| NPAS3 | | | | + | | | |
| SCGB2A2 | | | | + | | | |
| SSX3 | | | | + | + | + | |

Hierarchical categorization of 24 different original HRneg or Tneg prognostic gene candidates produced two 1^{st} (CLIC5, CXCL13), five 2^{nd} (PRTN3, FLJ46061/RPS28, SSX3, ABO, RGS4), and seven 3^{rd} (ZNF3, HAPLN3, EXOC7, RFXDC2, PRRG3, MATN1, HRBL) level candidates for further evaluation by RT-PCR analysis using a larger set of untreated HRneg or Tneg breast cancers associated with long clinical follow-up (Guy's tumor set).

Accordingly, this invention provides methods for the diagnosis and prognostic evaluation of breast cancer subtypes HRneg and Tneg based on the differential expression of any of the genes found in Tables 1 and 2, in breast cancer cells. The markers can be used alone or in combinations of two or more, or as a panel or markers. In some embodiments, the markers can be used as a set selected from the group consisting of HRneg S1, HRneg S2, HRneg SHP, Tneg S1, Tneg S2, Tneg SHP, HRTCS, a combination thereof, and a subset of a combination thereof. The invention also provides kits for diagnosis or prognosis of breast cancer subtypes HRneg and Tneg comprising one or more of the markers. The invention also provides therapeutic modulator compounds, antibodies, and siRNAs complementary to a sequence of one or more of the markers for treatment of a subtype of breast cancer.

### A. DEFINITIONS

The term “marker” refers to a molecule (typically protein, nucleic acid, carbohydrate, or lipid) that is expressed in the cell, expressed on the surface of a cancer cell or secreted by a cancer cell in comparison to a normal cell, and which is useful for the diagnosis of cancer, for providing a prognosis, and for preferential targeting of a pharmacological agent to the cancer cell. Oftentimes, such markers are molecules that are differentially expressed, e.g., overexpressed or underexpressed in a breast cancer cell or other cancer cell in comparison to a normal cell, for instance, 1-fold over/under expression, 2-fold over/under expression, 3-fold over/under expression or more in comparison to a normal cell or a primary cancer (as opposed to a metastasized cancer). Further, a marker can be a molecule that is inappropriately synthesized in the cancer cell, for instance, a molecule that contains deletions, additions or mutations in comparison to the molecule expressed on a normal cell. A non-cancer cell is considered a normal cell.

It will be understood by the skilled artisan that markers may be used singly or in combination with other markers for any of the uses, e.g., diagnosis or prognosis of a breast cancer subtype, disclosed herein.

The term “HRneg (hormone receptor negative) breast cancer subtype” refers to breast cancers that express estrogen receptor (ER) and progesterone receptor (PR) at a low or undetectable level. A “Tneg (triple negative) breast cancer subtype” refers to breast cancers that express ER, PR, and HER2 (ERB2) at a low or undetectable level. The independent expression levels of ER, PR, and HER2 in breast cancers are generally bimodal, meaning that a certain percentage of breast cancers express ER, PR, and/or HER2 at a relatively high level, while another subset expresses at a relatively low level. Those of skill in the art will understand that HRneg and TRneg status can be determined using standard methods, such as immunoaffinity assays or polynucleotide-based assays specific for ER, PR, and HER2 (see, e.g., van de Vijver et al. (2002) N. Engl. J. Med. 347:1999-09).

Some marker sets of the invention include HRneg S1, HRneg S2, HRneg HP, Tneg S1, Tneg S2, Tneg HP, and HRTCS. These marker sets are defined in Table 1. An additional marker set includes the 14 Gene Profile (or the 14 Gene Finalists) which includes: CXCL13, HAPLN1, FLJ46061///RPS28, RGS4, SSX3, RFXDC2, EXOC7, CLIC5, ZNF3, PRRG3, ABO, PRTN3, HRBL, MATN.

As used herein, the term “providing a prognosis” refers to providing a prediction of the probable course and outcome of cancer. The methods can also be used to devise a suitable therapy for cancer treatment, and more preferably a suitable therapy for a subtype of breast cancer such as HRneg or Tneg, e.g., by indicating whether or not the cancer is still at a benign stage or if the cancer had advanced to a stage where aggressive therapy would be required.

As used herein, the terms “treatment,” “treating,” “prevention,” and “preventing” and like terms are not intended to be absolute terms. Treatment and prevention can refer to any delay in onset, amelioration of symptoms, improvement in patient survival, reduction of tumor growth, reduction in metastasis or colony formation, etc. The effect of treatment can be compared to an individual or pool of individuals not receiving the treatment, or to an untreated tissue in the same patient.

The terms “reduced” and “increased” and similar relative terms are used herein to refer to a reductions, increases, etc. relative to a control value. Those of skill in the art are capable of determining an appropriate control for each situation. For example, if compound is said to reduce expression of gene X, the level of gene X expression in the presence of the compound is lower than the level of gene X expression in the absence of the compound.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, lymph and tongue tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., breast, skin, colon, prostate, kidney, bladder, lymph node, liver, bone marrow, blood cell, etc.), the size and type of the tumor (e.g., solid, suspended, or blood), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

The terms “overexpress,” “overexpression” or “overexpressed” interchangeably refer to a protein or nucleic acid (RNA) that is transcribed or translated at a detectably greater level, usually in a cancer cell, in comparison to a normal cell. The term includes overexpression due to transcription, post transcriptional processing, translation, post-translational processing, cellular localization (e.g., organelle, cytoplasm, nucleus, cell surface), and RNA and protein stability, as compared to a normal cell. Overexpression can be detected using conventional techniques for detecting mRNA (i.e., RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

The terms “underexpress,” “underexpression” or “underexpressed” interchangeably refer to a protein or nucleic acid (RNA) that is transcribed or translated at a detectably lower level, usually in a cancer cell, in comparison to a normal cell, a nevi, or a primary cancer. The term includes underxpression due to transcription, post transcriptional processing, translation, post-translational processing, cellular localization (e.g., organelle, cytoplasm, nucleus, cell surface), and RNA and protein stability, as compared to a normal cell. Underexpression can be detected using conventional techniques for detecting mRNA (i.e., RT-PCR, PCR, hybridization) or proteins (i.e., ELISA, immunohistochemical techniques). Underexpression can be 10%, 20%, 30%, 40%, 50%, 60%, 70%, 80%, 90% etc. in comparison to a normal cell. In certain instances, underexpression is 1-fold, 2-fold, 3-fold, 4-fold or more lower levels of transcription or translation in comparison to a normal cell.

“Therapeutic treatment” and “cancer therapies” refers to chemotherapy, hormonal therapy, radiotherapy, immunotherapy, gene therapy, and biologic (targeted) therapy.

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site at ncbi.nlm.nih.gov/BLAST or the like). Such sequences are then said to be “substantially identical.” This definition also refers to, or may be applied to, the compliment of a test sequence. The definition also includes sequences that have deletions and/or additions, as well as those that have substitutions. Similarly, this definition also includes sequences having alternatively spliced exons and/or introns or that are transcribed from alternate start codons. As described below, the preferred algorithms can account for gaps and the like. Preferably, identity exists over a region that is at least about 25 amino acids or nucleotides in length, or more preferably over a region that is 50-100 amino acids or nucleotides in length, or most preferably over a region corresponding to the entire length of the polypeptide or nucleic acid molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)), or by structural alignment and visual inspection thereof.

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

An “oligonucleotide,” such as an oligonucleotide probe, generally refers to a relatively short polynucleotide sequence or polynucleotide fragment. Oligonucleotides are often designed to be complementary to a subsequence of a particular gene for use as primers or probes for detection assays. Oligonucleotides usually range from about 4-100 nucleic acids, e.g., 18-35 nucleic acids, in length.

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

An “antisense” polynucleotide is a polynucleotide that is substantially complementary to a target polynucleotide and has the ability to specifically hybridize to the target polynucleotide.

Ribozymes are enzymatic RNA molecules capable of catalyzing specific cleavage of RNA. The composition of ribozyme molecules preferably includes one or more sequences complementary to a target mRNA, and the well known catalytic sequence responsible for mRNA cleavage or a functionally equivalent sequence (see, e.g., U.S. Pat. No. 5,093,246). Ribozyme molecules designed to catalytically cleave target mRNA transcripts can also be used to prevent translation of subject target mRNAs.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of cancer antigens. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). See, e.g., Creighton, Proteins (1984).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The antibody can be conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” when referring to a protein, nucleic acid, antibody, or small molecule compound refers to a binding reaction that is determinative of the presence of the protein or nucleic acid, particularly a protein or nucleic acid listed in Table 1, often in a heterogeneous population of proteins or nucleic acids and other biologics. In the case of antibodies, under designated immunoassay conditions, a specified antibody may bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

The phrase “functional effects” in the context of assays for testing compounds that modulate a marker protein includes the determination of a parameter that is indirectly or directly under the influence of a marker protein such as any of the proteins listed in Table 1, e.g., a chemical or phenotypic effect such as altered transcriptional activity of any one of the genes listed in Table 1 or altered activity of the downstream effects of such proteins listed in Table 1 on cellular metabolism and growth. A functional effect therefore includes ligand binding activity, transcriptional activation or repression, the ability of cells to proliferate, expression in cells during breast cancer progression, and other characteristics of breast cancer cells, and particularly of HRneg and Tneg breast cancer cell subtypes. “Functional effects” include in vitro, in vivo, and ex vivo activities.

By “determining the functional effect” is meant assaying for a compound that increases or decreases a parameter that is indirectly or directly under the influence of a marker such as any of the markers listed in Table 1, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to increase or decrease cellular proliferation, apoptosis, cell cycle arrest, measuring changes in cell surface markers. Determination of the functional effect of a compound on breast cancer cell, and more preferably HRneg or Tneg subtype breast cancer cell, progression can also be performed using assays known to those of skill in the art such as metastasis of breast cancer cells by tail vein injection of breast cancer cells in mice. The functional effects can be evaluated by many means known to those skilled in the art, e.g., microscopy for quantitative or qualitative measures of alterations in morphological features, measurement of changes in RNA or protein levels for other genes expressed in breast cancer cells, measurement of RNA stability, identification of downstream or reporter gene expression (CAT, luciferase, β-gal, GFP and the like), e.g., via chemiluminescence, fluorescence, colorimetric reactions, antibody binding, inducible markers, etc.

“Inhibitors,” “activators,” and “modulators” of the markers are used to refer to activating, inhibitory, or modulating molecules identified using in vitro and in vivo assays of breast cancer subtype markers such as those listed in Table 1. “Inhibitors” or “antagonists” are compounds that, e.g., bind to, partially or totally block activity, decrease, prevent, delay activation, inactivate, desensitize, or down regulate the activity or expression of breast cancer subtype markers such as those listed in Table 1. “Activators” are compounds that increase, open, activate, facilitate, enhance activation, sensitize, agonize, or up regulate activity of breast cancer subtype markers such as those listed in Table 1. Inhibitors, activators, or modulators also include genetically modified versions of breast cancer subtype markers such as those listed in Table 1., e.g., versions with altered activity, as well as naturally occurring and synthetic ligands, antagonists, agonists, antibodies, peptides, cyclic peptides, nucleic acids, antisense molecules, ribozymes, RNAi molecules, small organic molecules and the like. Such assays for inhibitors and activators include, e.g., expressing breast cancer subtype markers such as those listed in Table 1 in vitro, in cells or cell extracts, applying putative modulator compounds, and then determining the functional effects on activity, as described above.

Samples or assays comprising breast cancer subtype markers such as those listed in Table 1 that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of breast cancer subtype markers such as those listed in Table 1 is achieved when the activity value relative to the control is about 80%, preferably 50%, more preferably 25-0%. Activation of breast cancer subtype markers such as those listed in Table 1 is achieved when the activity value relative to the control (untreated with activators) is 110%, more preferably 150%, more preferably 200-500% (i.e., two to five fold higher relative to the control), more preferably 1000-3000% higher.

The term “test compound,” “test agent,” “drug candidate,” or “modulator” or grammatical equivalents as used herein describes any molecule, either naturally occurring or synthetic, e.g., protein, oligopeptide (e.g., from about 5 to about 25 amino acids in length, e.g., 10 to 20 or 12 to 18 amino acids in length, or 12, 15, or 18 amino acids in length), small organic molecule, polysaccharide, peptide, circular peptide, lipid, fatty acid, siRNA, polynucleotide, oligonucleotide, etc., to be tested for the capacity to directly or indirectly modulate breast cancer subtype markers such as those listed in Table 1. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

### B. DIAGNOSTIC AND PROGNOSTIC METHODS

The present invention provides methods of diagnosing or providing prognosis of breast cancer subtypes by detecting the expression of markers highly expressed in breast cancer subtype cells at different stages of malignancy. Diagnosis involves determining the level of a polypeptide or nucleic acid, such as for the marker genes listed in Table 1, in a patient or patient sample and then comparing the level to a control baseline or range. Typically, the baseline value is representative of levels of the polynucleotide or nucleic acid in a person not suffering from breast cancer or a patient not suffering from a specific subtype of breast cancer, as measured using a biological sample such as a breast tissue biopsy, a skin biopsy, or other appropriate control. Variation of levels of a polynucleotide or nucleic acid of the invention from the baseline range (either up or down) indicates that the patient has a specific subtype of breast cancer or is at risk of developing a specific subtype of breast cancer, depending on the marker or markers used.

A control baseline or range value can be obtained by statistically compiling and/or averaging a number of control samples. Control samples, which often include normal, non-cancer cells, can comprise breast or non-breast tissue. For example, in the case of some genes, expression is undetectable in normal breast tissue. Thus, it can be useful to use another tissue, for which expression of the gene in question is standardized and detectable. The control sample can be obtained from the same individual, e.g., where differences between individuals are significant, or can be obtained from a different individual. Design of appropriate controls is understood by those of skill in the art, and can vary depending on which genes are tested, and which breast cancer subtype is tested.

In some embodiments, a test sample from a patient (e.g., a breast biopsy) will be compared to a control sample where the outcome is known. For example, a control sample can be a tumor sample from a patient or set of patients with non-nodal HRneg or Tneg breast cancer that progressed after a known period of time, or did not progress. Again, it will be understood that one of skill will be able to determine an appropriate control based on the particular circumstances of the test.

### 1. Immunoaffinity-Based Methods

Antibody reagents can be used in assays to detect expression levels of marker genes, such as those found in Table 1, in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to nucleic acids can be detected directly or indirectly, as described below.

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

### 2. Nucleic Acid-Based Methods

Alternatively, nucleic acid binding molecules such as probes, oligonucleotides, oligonucleotide arrays, and primers can be used in assays to detect differential RNA expression in patient samples, e.g., RT-PCR. In some embodiments, RT-PCR is used according to standard methods known in the art. In some embodiments, PCR assays such as Taqman® assays available from, e.g., Applied Biosystems, can be used to detect nucleic acids and variants thereof. In some embodiments, qPCR and nucleic acid microarrays can be used to detect nucleic acids. Reagents that bind to selected cancer biomarkers can be prepared according to methods known to those of skill in the art or purchased commercially (e.g., from Affymetrix).

In some embodiments, primers (for RT-PCR, for example) or probes (e.g., for microarray analysis) are designed to specifically detect a marker of the invention, i.e., “gene-unique.” In some embodiments, the primers or probes are designed to detect a particular variant, e.g., a splice variant or allelic variant, of a given marker gene. Such sequences are said to be “transcript-unique.” In some embodiments, the primers or probes are designed to detect all variants of a given marker gene.

For example, the marker genes RGS4, ZNF3, and RPS28 have a number of splice variants. SSX3 has paralogs on the X chromosome. Thus, primers or probes can be designed or selected to detect a shared sequence, e.g., a common exon or UTR sequence. If a commercially available or preset detection assay is used (e.g., from Affymetrix), one can determine beforehand which variant sequences are detected by the preset sequences, or if the preset sequences are specific for the marker genes.

Analysis of nucleic acids can also be achieved using routine techniques such as Southern or Northern analysis, sequence analysis, microarrays, or any other methods based on hybridization between complementary nucleic acid sequences (e.g., slot blot hybridization). Applicable PCR amplification techniques are described in, e.g., Ausubel et al. and Innis et al., supra. General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression From Experiments to Data Analysis and Modeling,” Cambridge University Press, 2002.

Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell. Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Other methods for detecting nucleic acid variants include, e.g., the INVADER® assay from Third Wave Technologies, Inc., restriction fragment length polymorphism (RFLP) analysis, allele-specific oligonucleotide hybridization, a heteroduplex mobility assay, single strand conformational polymorphism (SSCP) analysis, single-nucleotide primer extension (SNUPE) and pyrosequencing.

### 3. Labels and Detectable Moieties

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Direct labels include fluorescent or luminescent tags, metals, dyes, radionucleotides, and the like, attached to the antibody. An antibody labeled with iodine-125 (¹²⁵I) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, Oregon Green™, rhodamine, Texas red, tetrarhodimine isothiocynate (TRITC), Cy3, Cy5, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

Useful physical formats comprise surfaces having a plurality of discrete, addressable locations for the detection of a plurality of different markers. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection.

Analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

The antibodies or nucleic acid probes of the invention can be applied to sections of patient biopsies immobilized on microscope slides. The resulting antibody staining or in situ hybridization pattern can be visualized using any one of a variety of light or fluorescent microscopic methods known in the art.

### C. COMPOSITIONS, KITS, AND INTEGRATED SYSTEMS

The invention provides compositions, kits and integrated systems for practicing the assays described herein using antibodies specific for the polypeptides or nucleic acids specific for the polynucleotides of the invention.

Kits for carrying out the diagnostic assays of the invention typically include a probe that comprises an antibody or nucleic acid sequence that specifically binds to polypeptides or polynucleotides of the invention, and a label for detecting the presence of the probe. The kits may include several antibodies or polynucleotide sequences encoding polypeptides of the invention, e.g., a cocktail of antibodies that recognize at least two marker proteins listed in Table 1. In other embodiments, these cocktails may include antibodies that recognize at least 3, 4, 5, 6, 7, 8, 9, 10, 11, 12, 13, 14, 15, 16, 17, 18, 19, 20, 21, 22, or 23 of the marker genes in Table 1 in any combination. In some embodiments, the cocktails include antibodies that recognize all of the marker genes listed in Table 1.

### D. IN VIVO IMAGING

The various markers of the invention also provide reagents for in vivo imaging such as, for instance, the imaging of metastasis of breast cancer subtypes to regional lymph nodes using labeled regents that detect one or more of the proteins or nucleic acids listed in Table 1. In vivo imaging techniques may be used, for example, as guides for surgical resection or to detect the distant spread of metastatic cells. For in vivo imaging purposes, reagents that detect the presence of one or more of the markers listed in Table 1, such as antibodies, may be labeled with a positron-emitting isotope (e.g., 18F) for positron emission tomography (PET), gamma-ray isotope (e.g., 99 mTc) for single photon emission computed tomography (SPECT), a paramagnetic molecule or nanoparticle (e.g., Gd3+ chelate or coated magnetite nanoparticle) for magnetic resonance imaging (MRI), a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

Furthermore, such reagents may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720, U.S. Pat. No. 5,227,487, and U.S. Pat. No. 5,543,295.

Other fluorescent labels suitable for use in the practice of this invention include a fluorescein dye. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. No. 6,008,379, U.S. Pat. No. 5,750,409, U.S. Pat. No. 5,066,580, and U.S. Pat. No. 4,439,356. A cargo portion C may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and other rhodamine dyes. Other rhodamine dyes can be found, for example, in U.S. Pat. No. 6,080,852, U.S. Pat. No. 6,025,505, U.S. Pat. No. 5,936,087, U.S. Pat. No. 5,750,409. A cargo portion C may include a cyanine dye, such as, for example, Cy3, Cy3B, Cy3.5, Cy5, Cy5.5, Cy 7. Phosphorescent compounds including porphyrins, phthalocyanines, polyaromatic compounds such as pyrenes, anthracenes and acenaphthenes, and so forth, may also be used.

Reagents such as antibodies may include a radioactive moiety, for example a radioactive isotope such as ²¹¹At, ¹³¹I, ¹²⁵I, ⁹⁰Y, ¹⁸⁶Re, ¹⁸⁸Re, ¹⁵³Sm, ²¹²Bi, ³²P, radioactive isotopes of Lu, and others.

### E. METHODS OF DIAGNOSIS AND MONITORING BREAST CANCER

A number of conventional methods are used to diagnose and monitory breast cancer. Standard screening methods include breast self exams and mammograms. If an abnormality is detected, a biopsy is usually taken to follow up. The biopsy tissue can be examined by a pathologist to determine if cancer cells are present.

After positive diagnosis, monitoring techniques include breast self exams, mammograms and biopsies. Blood counts can be taken, as cancer or chemotherapeutic therapies can affect the number of platelets and red and white blood cells. In some cases, blood chemistry can be analyzed to determine if bone, kidney, or liver function is affected. Imaging techniques can also be used to monitor a breast cancer patient. These include MRI, CT scans, and X-ray. Digital imaging techniques, such as digital tomosynthesis, can also be used, as these avoid the drawbacks of mammographies (discomfort, overlapping breast tissue masking abnormalities). Ultrasound is also used, and is considered useful for determining if an abnormality is solid (such as a benign fibroadenoma or cancer) or fluid-filled (such as a benign cyst).

Common techniques for detecting the BRCA or HER2 status of a patient include fluorescence in situ hybridization (FISH) and immunofluorescence techniques. As explained above, HER2 is correlated with breast cancer in a subset of breast cancer patients. These techniques can also be used to detect markers associated with breast cancer or breast cancer subsets, including ER, PR, HER2 (c-ERB2) and the markers listed in Table 1.

### F. THERAPY MODIFICATION AND TREATMENT OPTIONS

The invention provides methods of adjusting therapy for breast cancer based on a prognosis obtained using the HRneg and Tneg markers in Table 1. Currently, almost all newly diagnosed patients are treated with adjuvant combination chemotherapy, such as CMF chemotherapy or Anthracycline-based chemotherapy. HRneg and Tneg breast cancers are vary considerably in metastatic potential, however, indicating that in many cases, aggressive chemotherapy is unnecessary or misdirected.

The CMF chemotherapy regimen includes a combination of cyclophosphamide, methotrexate, and 5-fluorouracil (abbreviated 5-FU). This combination can be given into a vein (intravenous, called IV CMF), or with oral cyclophosphamide plus IV methotrexate and 5-FU (termed oral or classic CMF). Most doctors consider oral CMF to be more effective than the all-IV version. Anthracycline-based chemotherapy (using, e.g., doxorubicin [Adriamycin®] or epirubicin [Ellence®]), can be combined with a taxane (paclitaxel [Taxol®] or docetaxel [Taxotere®]). Taxanes are now routinely included as a component of the adjuvant chemotherapy regimen for women with node-positive breast cancer, and for some high-risk node-negative breast cancers. A popular type of anthracycline- and taxane-containing adjuvant chemotherapy called dose-dense therapy. Radiation therapy can also be used alone or in combination with chemotherapy.

While these therapies offer improved outcomes in many breast cancer patients, they result in unpleasant, and sometimes dangerous, side effects. Side effects include hair loss, nausea, diarrhea, neurologic toxicity, weight gain, fatigue, impaired memory or concentration, hot flashes, premature menopause, heart disease, and leukemia.

Thus, in some embodiments, the methods of the invention can be used to avoid or reduce unnecessary therapies. Further, a patient can be monitored using the methods described herein to determine if the therapeutic regimen should be modified. Monitoring can include detecting the level of at least one of the markers listed in Table 1, or monitoring breast tissue and lymph nodes, as will be understood in the art.

For example, a newly diagnosed breast cancer patient can be tested for expression of the marker genes listed in Table 1, or a subset thereof. If the gene expression profile correlates with good prognosis, less aggressive therapeutic regimen can be pursued, e.g., delayed treatment or lower initial dose than what would normally be prescribed.

In some embodiments, chemotherapy and/or radiation can be combined with modulators that target the marker genes listed in Table 1. For example, modulators of at least one of the marker genes listed in Table 1 can be combined with chemotherapy. In some embodiments, the chemotherapeutic agent can be administered at a dose that would be ineffective in the absence of the modulator compound. Such methods are useful for reducing the likelihood of side effects from either therapeutic agent. Methods of identifying and using modulators of the marker genes listed in Table 1, including compounds, antibodies and nucleic acids, are described below.

### G. METHODS TO IDENTIFY MODULATOR COMPOUNDS

A variety of methods may be used to identify compounds that prevent or treat breast cancer (e.g., HRneg or Tneg) progression. Typically, an assay that provides a readily measured parameter is adapted to be performed in the wells of multi-well plates in order to facilitate the screening of members of a library of test compounds as described herein. Thus, in one embodiment, an appropriate number of cells can be plated into the cells of a multi-well plate, and the effect of a test compound on the expression of one or more marker genes, such as those listed in Table 1, can be determined.

The compounds to be tested can be any small chemical compound, or a macromolecule, such as a protein, sugar, nucleic acid or lipid. Typically, test compounds will be small chemical molecules and peptides. Essentially any chemical compound can be used as a test compound in this aspect of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In some embodiments, high throughput screening methods are used which involve providing a combinatorial chemical or peptide library containing a large number of potential therapeutic compounds. Such “combinatorial chemical libraries” or “ligand libraries” are then screened in one or more assays, as described herein, to identify those library members (particular chemical species or subclasses) that display a desired characteristic activity. In this instance, such compounds are screened for their ability to modulate the expression or activity of one or more of the marker genes listed in Table 1.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries are well known to those of skill in the art. Such combinatorial chemical libraries include, but are not limited to, peptide libraries (see, e.g., U.S. Pat. No. 5,010,175, Furka, Int. J. Pept. Prot. Res., 37:487-493 (1991) and Houghton et al., Nature, 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., PNAS USA, 90:6909-6913 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-9218 (1992)), analogous organic syntheses of small compound libraries (Chen et al., J. Amer. Chem. Soc., 116:2661 (1994)), oligocarbamates (Cho et al., Science, 261:1303 (1993)), and/or peptidyl phosphonates (Campbell et al., J. Org. Chem., 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100-about 1500 different compounds. It is possible to assay many plates per day; assay screens for up to about 6,000, 20,000, 50,000, or 100,000 or more different compounds is possible using the integrated systems of the invention.

### H. MODULATOR ANTIBODIES

Antibodies that specifically bind to the marker genes listed in Table 1 can be used in the methods of the invention. For preparation of suitable antibodies and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)).

In some embodiments, the antibody reduces activity of the marker gene. The antibodies of the invention can be raised against full length proteins or fragments, or produced recombinantly. Any number of techniques can be used to determine antibody binding specificity. See, e.g., Harlow & Lane, Antibodies, A Laboratory Manual (1988) for a description of immunoassay formats and conditions that can be used to determine specific immunoreactivity of an antibody.

In some embodiments, the antibody is a polyclonal antibody. Methods of preparing polyclonal antibodies are known to the skilled artisan (e.g., Harlow & Lane, Antibodies, A Laboratory manual (1988); Methods in Immunology). Polyclonal antibodies can be raised in a mammal by one or more injections of an immunizing agent and, if desired, an adjuvant. The immunizing agent in this case includes a marker protein, or fragment thereof, e.g., an extracellular domain.

In some embodiments, the antibody is a monoclonal antibody. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent (e.g., a marker fragment) to elicit lymphocytes that produce or are capable of producing antibodies that will specifically bind to the immunizing agent. Alternatively, the lymphocytes may be immunized in vitro.

Human monoclonal antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

### I. NUCLEIC ACID MODULATORS

Depending on whether a particular marker gene is expressed at a higher or lower level relative to baseline, nucleic acid techniques can be used to reduce or increase expression of the marker.

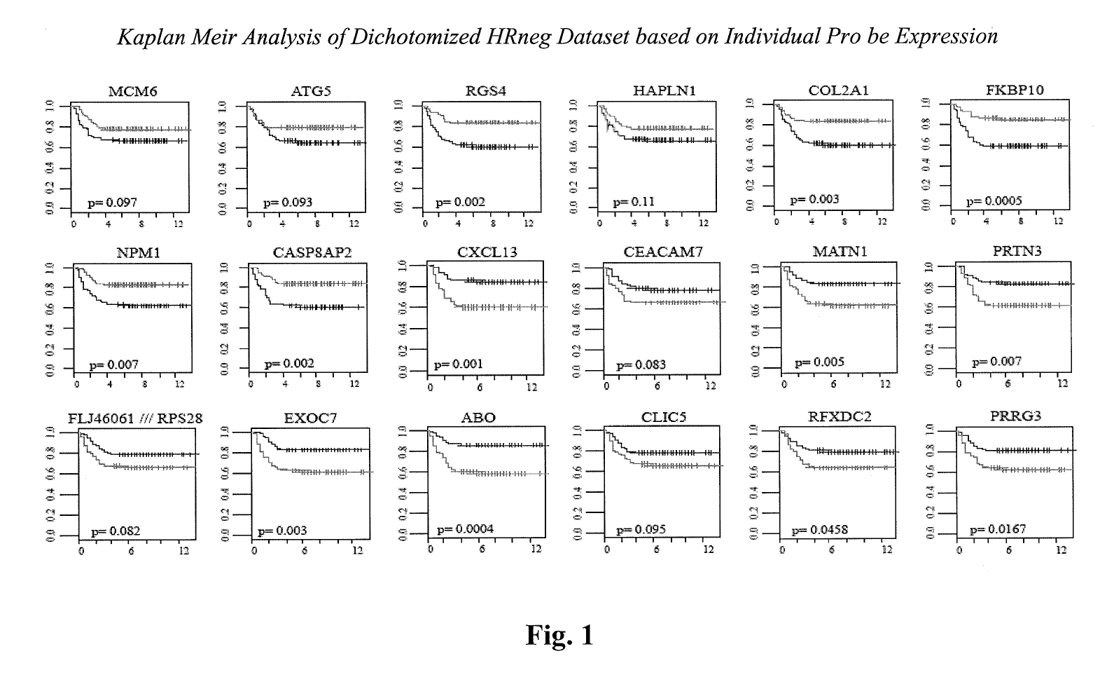

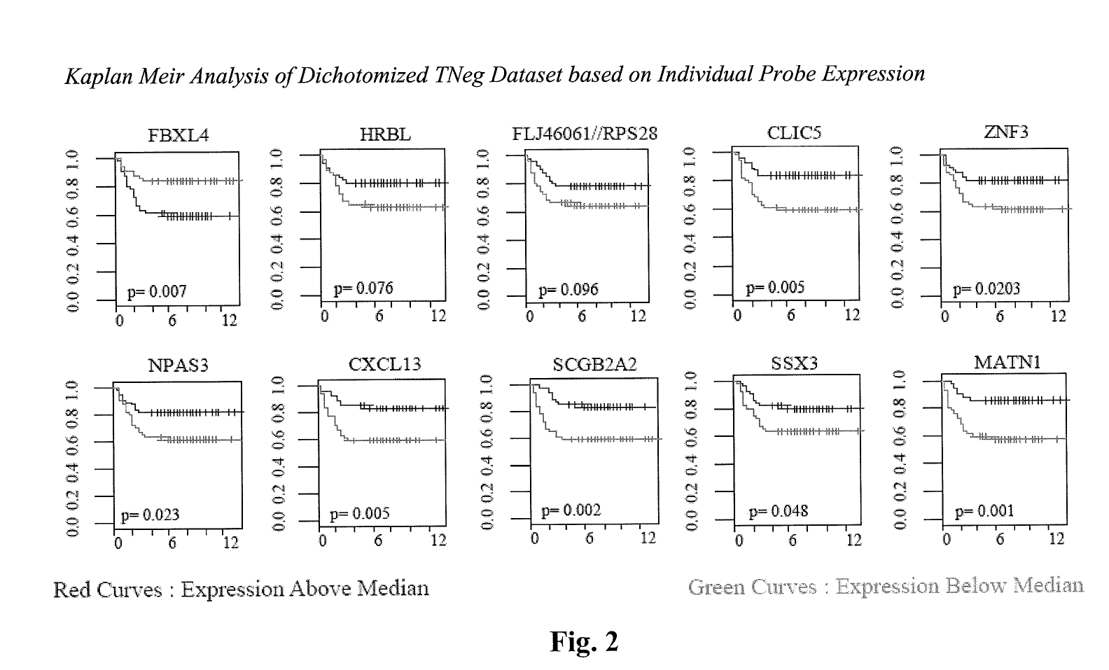

In some embodiments, therefore, it is desirable to upregulate expression of a marker gene, for example, for markers with above-median expression levels that correlate with better survival rate. The relative expression of the marker genes of Table 1 in HRneg and Tneg breast cancers are described, e.g., in the examples and FIGS. 1 and 2. The accession numbers of each of the marker genes in Table 1 are provided in Table 3.

Thus, the coding sequence for a particular marker can be introduced into a cell, e.g., a breast cancer cell or normal breast tissue, as described below. In some embodiments, the coding sequence will be reflect a particular allelic variant or splice variant of the marker gene.

Alternatively, it can be desirable to inhibit the expression of a particular marker gene in Table 1. A variety of nucleic acids, such as antisense nucleic acids, siRNAs or ribozymes, can be used to for this purpose. Ribozymes that cleave mRNA at site-specific recognition sequences can be used to destroy target mRNAs, particularly through the use of hammerhead ribozymes. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA.

With regard to antisense, siRNA or ribozyme oligonucleotides, phosphorothioate oligonucleotides can be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Coding sequences and inhibitory oligonucleotides can be delivered to a cell by direct transfection or transfection and expression via an expression vector. Appropriate expression vectors include mammalian expression vectors and viral vectors, into which has been cloned the desired polynucleotide sequence with the appropriate regulatory sequences including a promoter to result in expression of the RNA (coding or antisense) in a host cell. Suitable promoters can be constitutive or development-specific promoters. Transfection delivery can be achieved by liposomal transfection reagents, known in the art (e.g., Xtreme transfection reagent, Roche, Alameda, Calif.; Lipofectamine formulations, Invitrogen, Carlsbad, Calif.). Delivery mediated by cationic liposomes, by retroviral vectors and direct delivery are efficient. Another possible delivery mode is targeting using antibody to cell surface markers for the target cells.

For transfection, a composition comprising one or more nucleic acid molecules (within or without vectors) can comprise a delivery vehicle, including liposomes, for administration to a subject, carriers and diluents and their salts, and/or can be present in pharmaceutically acceptable formulations. Methods for the delivery of nucleic acid molecules are described, for example, in Gilmore, et al., Curr Drug Delivery (2006) 3:147-5 and Patil, et al., AAPS Journal (2005) 7:E61-E77. Delivery of siRNA molecules is also described in several U.S. patent Publications, including for example, 2006/0019912; 2006/0014289; 2005/0239687; 2005/0222064; and 2004/0204377. Nucleic acid molecules can be administered to cells by a variety of methods known to those of skill in the art, including, but not restricted to, encapsulation in liposomes, by iontophoresis, by electroporation, or by incorporation into other vehicles, including biodegradable polymers, hydrogels, cyclodextrins (see, for example Gonzalez et al., 1999, Bioconjugate Chem., 10, 1068-1074; Wang et al., WO03/47518 and WO 03/46185), poly(lactic-co-glycolic)acid (PLGA) and PLCA microspheres (see for example U.S. Pat. No. 6,447,796 and US Patent Application Publication No. 2002/130430), biodegradable nanocapsules, and bioadhesive microspheres, or by proteinaceous vectors (O'Hare and Normand, International PCT Publication No. WO 00/53722). In another embodiment, the nucleic acid molecules of the invention can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

Examples of liposomal transfection reagents of use with this invention include, for example: CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); Cytofectin GSV, 2:1 (M/M) liposome formulation of a cationic lipid and DOPE (Glen Research); DOTAP (N-[1-(2,3-dioleoyloxy)-N,N,N-tri-methyl-ammoniummethylsulfate) (Boehringer Manheim); Lipofectamine, 3:1 (M/M) liposome formulation of the polycationic lipid DOSPA and the neutral lipid DOPE (GIBCO BRL); and (5) siPORT (Ambion); HiPerfect (Qiagen); X-treme GENE (Roche); RNAicarrier (Epoch Biolabs) and TransPass (New England Biolabs).

In some embodiments, the polynucleotide construct is delivered into the cell via a mammalian expression vector. For example, mammalian expression vectors suitable for siRNA expression are commercially available, for example, from Ambion (e.g., pSilencer vectors), Austin, Tex.; Promega (e.g., GeneClip, siSTRIKE, SiLentGene), Madison, Wis.; Invitrogen, Carlsbad, Calif.; InvivoGen, San Diego, Calif.; and Imgenex, San Diego, Calif. Typically, expression vectors for transcribing siRNA molecules will have a U6 promoter.

In some embodiments, the polynucleotide construct is delivered into cells via a viral expression vector. Viral vectors suitable for delivering such molecules to cells include adenoviral vectors, adeno-associated vectors, and retroviral vectors (including lentiviral vectors). For example, viral vectors developed for delivering and expressing siRNA oligonucleotides are commercially available from, for example, GeneDetect, Bradenton, Fla.; Ambion, Austin, Tex.; Invitrogen, Carlsbad, Calif.; Open BioSystems, Huntsville, Ala.; and Imgenex, San Diego, Calif.

### J. PHARMACEUTICAL COMPOSITIONS AND ADMINISTRATION

The agents as described herein (e.g., a modulator of a marker gene listed in Table 1, and combination therapies) can be administered to a human patient in accord with known methods. Information regarding pharmaceutical formulation and administration are detailed in Remington: The Science and Practice of Pharmacy, Gennaro, ed., Mack Publishing Co., Easton, Pa., 19th ed., 1995.

The compositions can be administered for therapeutic or prophylactic treatments. In therapeutic applications, compositions are administered to a patient suffering from breast cancer in a “therapeutically effective dose.” Amounts effective for this use will depend upon the mode of administration (e.g., oral, topical, parenteral, intravenous), severity of the disease, the general state of the patient's health, and the patient's age, weight, and pharmacological profile. Single or multiple administrations of the compositions may be administered depending on the dosage and frequency as required and tolerated by the patient. A “patient” or “subject” for the purposes of the present invention includes both humans and other animals, particularly mammals. Thus the methods are applicable to both human therapy and veterinary applications.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that oral administration requires protection from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art. Compositions for topical administration are also included, e.g., creams, powders (e.g., to be rehydrated), gels, sprays, etc.

Pharmaceutical formulations of the present invention can be prepared by mixing an agent having the desired degree of purity with optional pharmaceutically acceptable carriers, excipients or stabilizers. Such formulations can be lyophilized formulations or aqueous solutions. Acceptable carriers, excipients, or stabilizers are nontoxic to recipients at the dosages and concentrations used. Acceptable carriers, excipients or stabilizers can be acetate, phosphate, citrate, and other organic acids; antioxidants (e.g., ascorbic acid), preservatives, low molecular weight polypeptides; proteins, such as serum albumin or gelatin, or hydrophilic polymers such as polyvinylpyllolidone; and amino acids, monosaccharides, disaccharides, and other carbohydrates including glucose, mannose, or dextrins; chelating agents; and ionic and non-ionic surfactants (e.g., polysorbate); salt-forming counter-ions such as sodium; metal complexes (e.g. Zn-protein complexes); and/or non-ionic surfactants.

The formulation may also provide additional active compounds, including, chemotherapeutic agents, cytotoxic agents, cytokines, growth inhibitory agent, and anti-hormonal agent. The active ingredients may also prepared as sustained-release preparations (e.g., semi-permeable matrices of solid hydrophobic polymers (e.g., polyesters, hydrogels (for example, poly (2-hydroxyethyl-methacrylate), or poly (vinylalcohol)), polylactides. The antibodies and immunocongugates may also be entrapped in microcapsules prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macro emulsions.

In the case of antibodies, e.g., an antibody specific for a marker gene listed in Table 1, aqueous solutions are commonly administered by injection, e.g., intravenous administration, as a bolus or by continuous infusion over a period of time. Alternatively administration can be intramuscular, intraperitoneal, intracerobrospinal, subcutaneous, intra-articular, intrasynovial, intrathecal, oral, topical, or inhalation routes. Intravenous or subcutaneous administration of the antibody is preferred. The administration may be local or systemic.

The compositions for administration will commonly comprise an agent as described herein (e.g., a modulator of a marker gene listed in Table 1 and combination therapies) dissolved in a pharmaceutically acceptable carrier, preferably an aqueous carrier. A variety of aqueous carriers can be used, e.g., buffered saline and the like. These solutions are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agents in these formulations can vary widely, and will be selected primarily based on fluid volumes, viscosities, body weight and the like in accordance with the particular mode of administration selected and the patient's needs.

Thus, a typical pharmaceutical composition for intravenous administration will vary according to the agent. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The pharmaceutical preparation is preferably in unit dosage form. In such form the preparation is subdivided into unit doses containing appropriate quantities of the active component(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

In some cases, the pharmaceutical compositions are used in combination with surgery, chemotherapy, or radiation therapies. For example, a pharmaceutical composition of the present invention can be administered directly to a surgical site to reduce the likelihood of metastasis or recurrence.

Although specific embodiments of the invention have been described herein for purposes of illustration, various modifications can be made without deviating from the spirit and scope of the invention. Accordingly, the invention is not limited to the specific embodiments disclosed. All publications, patents, and patent applications cited herein are incorporated by reference in their entireties for all purposes.

### EXAMPLES

### A. Example 1

135 untreated, node-negative (NO), ER-negative primary breast cancers (HRneg) were identified from published studies which used the Affymetrix U133A microarray platform (Wang et al., 2005, GSE2034; Minn et al., 2007, GSES327). Array data was log2 transformed. Based on the cumulative distribution of mean-centered, log2 transformed ERBB2 mRNA transcript level (Probe Set ID 216836_s_at), a subset of 108 cases were identified as Tneg.

TABLE 2
Number of tumors identified as HRneg or Tneg

| | HRneg | | Tneg | |
|---|---|---|---|---|
| | Metastatic | | Metastatic | |
| | Events | Censored | Events | Censored |
| Wang et al cases | 27 | 50 | 21 | 39 |
| Minn et al cases | 11 | 47 | 10 | 38 |

The training dataset was subdivided by data source (Wang et at and Minn et at cases). Using PAM, ˜300 top discriminating probes between metastatic and non-metastatic cases were identified from each subset. Probes commonly selected from both subsets, with consistent directionality in the PAM importance score, were included in the next phase of the analysis.

A minimum variation filter was applied to exclude probes that did not have at least 10% observations exhibiting a two-fold change from mean probe expression. To adjust for variation between sources, data was median-centered and scaled by the standard deviation independently within each data source. For 100 iterations, the transformed data was randomly subdivided into training and test groups, balanced for the number of metastatic cases. Univariate Cox analysis was performed; and a Cox p-value was calculated based on the average Wald statistic over the iterations. Probes with a Cox p-value <0.01 and the same sign Cox coefficient in >80% of all paired training and test groups were included in the next phase of the analysis.

A multivariate Cox model was constructed from the probes selected from PAM and the Iterative Sampling algorithm. Probes with consistent directionality of correlation with survival in both univariate and multivariate Cox analysis were selected as candidate prognostic markers.

Based on the expression of a given candidate probe set, the Summation Index is calculated as follows:

$${\sum }_{i P}^{ } {x}_{i} - {\sum }_{i N}^{ } {x}_{i}$$

where x is the adjusted expression (median-centered and scaled to the standard deviation), i is a gene indicator, [symbol] signifies “contained in”, and P and N are the set of probes with positive and negative Cox coefficient respectively.

Datasets were dichotomized based on the median expression of candidates, where tumors with above median probe expression deemed “High” expressors and tumors with below median probe expressor deemed “Low” expressors. Similarly, dichotomization based on the Summation Index was also performed. For the Summation Index, an optimal cut-point for dataset dichotomization was identified using a modified Log-Rank statistic. Kaplan Meir analysis was performed to assess differences in survival between High vs. Low expressors; and the Log Rank test was used to estimate the significance in curve separation.

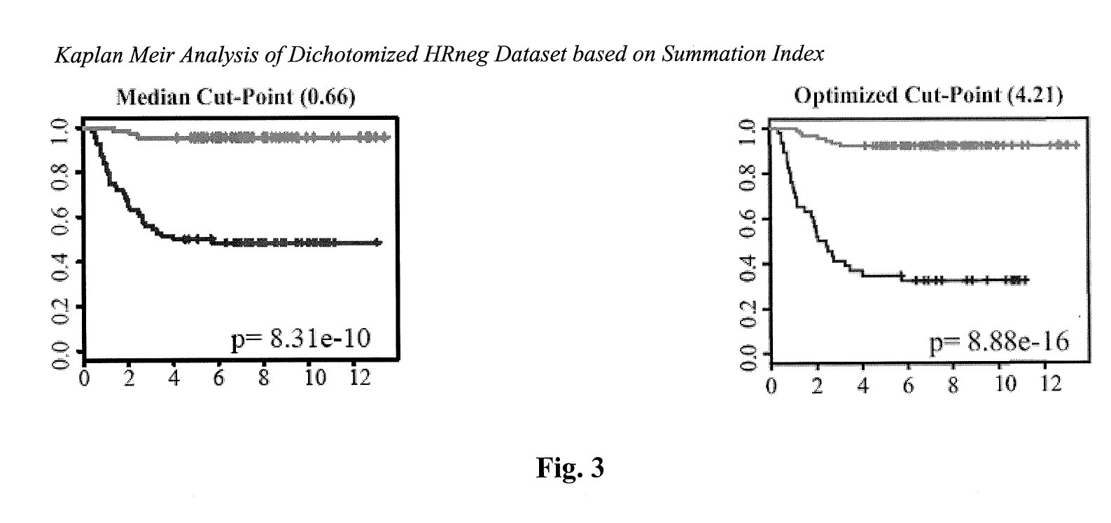

As a result of the analysis, 18 unique genes were identified as HRneg prognostic candidates. Kaplan Meier analyses based on individual probe expression for these 18 genes are shown in FIG. 1. A summation index of these markers indicates that they are prognostic in the HRneg dataset, as shown in the Kaplan-Meier analysis presented in FIG. 3.

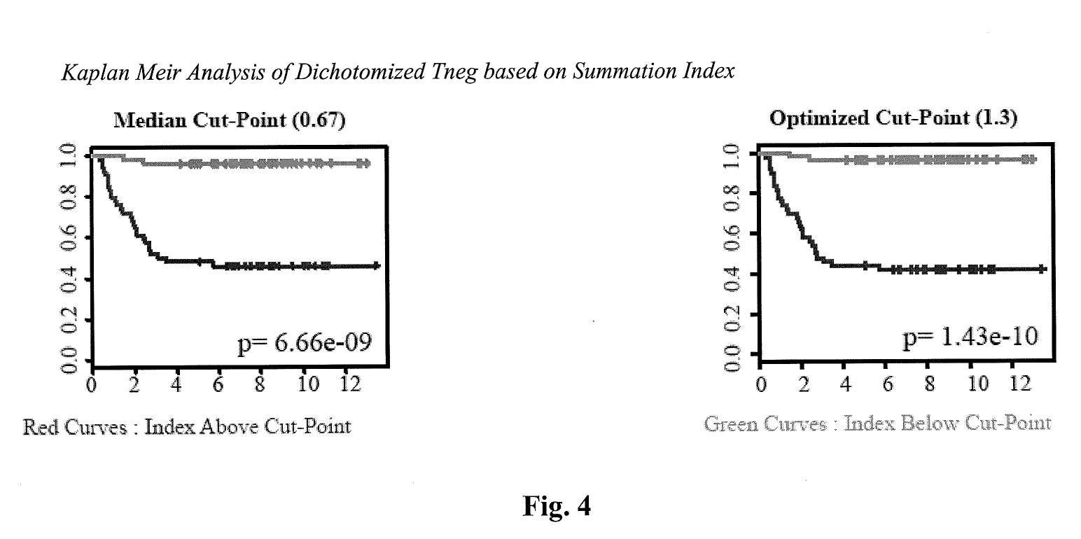

Furthermore, 10 unique genes were identified as Tneg candidate genes. Kaplan Meier analyses based on individual probe expression for these 10 genes are shown in FIG. 2. There is a strong trend for prognostic significance of the summation index calculated based on the expression of these markers in Tneg cases at median cut-point, as shown in FIG. 4.

Four of the identified candidate genes; FLJ46061/RPS28, CLIC5, CXCL13, and MATN1, were common to both prognostic sets. When these four candidate genes were used in a summation index, they were better able to predict metastasis-free survival than as single gene predictors.

### B. Example 2

Sixty-four untreated, NO HRneg primary breast cancers (24 metastastic cases) similarly analyzed using the Affymetrix platform were identified from the TRANSBIG multicenter validation series (Desmedt et al. 2007, GSE7390). Expression measures were generated using the RMA algorithm in Bioconductor R. Based on the cumulative distribution of the mean-centered ERBB2 transcript level (Probe 216836_s_at), a subset of 46 cases (23 metastasis) were identified as Tneg.

Univariate Cox analysis was performed based on the prioritization dataset. Candidates with Cox coefficients bearing the same sign as in the original Cox analysis of the training data set are deemed higher priority candidates and included in the next phase of the analysis.

Multi-variate Cox analysis was performed based on the priorization dataset. Genes with Cox coefficients bearing the same sign as in the original Cox analysis of the training data set are deemed highest priority candidates. Summation Index was computed and Kaplan Meir Analysis was performed.

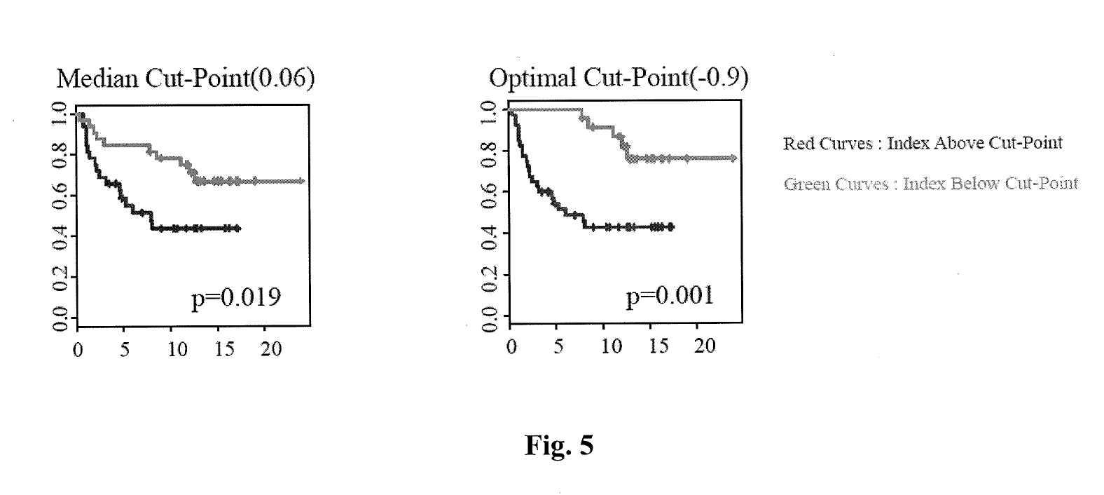

As a result of this analysis, 11 unique genes were selected as higher priority HRneg prognostic candidates (RGS4; HAPLN1; CXCL13; MATN1; PRTN3; FLJ46061///RPS28; EXOC7; ABO; CLIC5; RFXDC2; PRRG3). Furthermore, 8 of these genes (HAPLN1; CXCL13; PRTN3; FLJ46061///RPS28; EXOC7; CLIC5; RFXDC2; PRRG3) were selected as the Highest Priority Candidates. A summation index, calculated based on the expression of highest priority candidates, is prognostic in the HRneg prioritization dataset, as shown in the Kaplan-Meier analysis presented in FIG. 5.

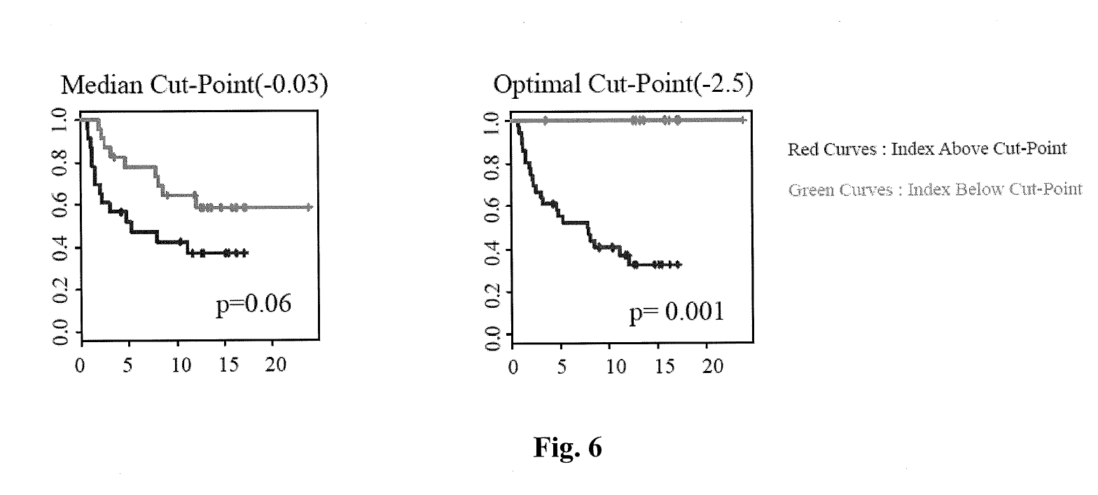

Similarly, this study identified 6 unique genes as higher priority Tneg prognostic candidates (HRBL; CLIC5, ZNF3, CXCL13, SSX3, MATN1). 5 of these genes were selected as the Highest Priority Tneg prognostic markers, excluding HRBL. There is a strong trend for prognostic significance of the summation index calculated based on the expression of highest priority probes in Tneg cases at median cut-point, as shown in FIG. 6. Further, at the optimal cut-point the survival difference between tumors with “High” vs “Low” summation index is statistically significant.

### C. Example 3

37 untreated, N0 HRneg tumors were selected from the NKI study (Netherlands Cancer Institute; see Van de Vijver et al. 2002) analyzed using the Agilent platform (13 metastastic cases). Based on the adjusted expression of 6 higher priority candidates found on this array platform (MATN1, ABO, RGS4, PRTN3, CLIC5, RPS28), a summation index was computed; and Kaplan Meir analysis was performed.

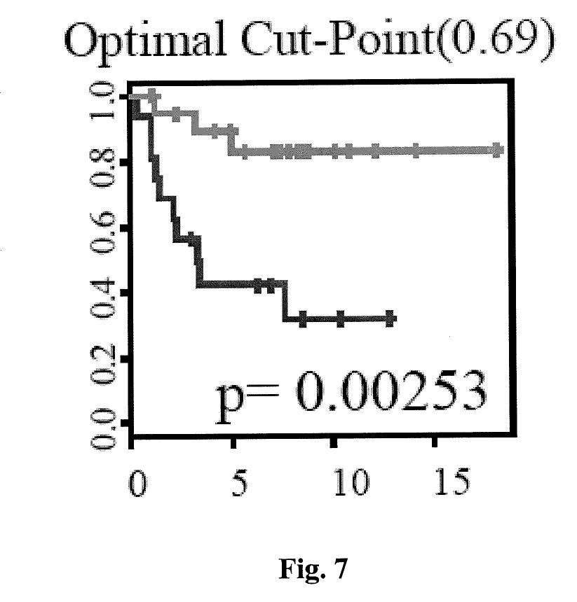

The result of the Kaplan-Meier analysis was that the summation index calculated based on expression of six higher priority candidates is prognostic in the NKI HRneg tumor set, as shown in FIG. 7.

Therefore, hierarchical categorization of 24 different original HRneg or Tneg prognostic gene candidates produced two 1st (CLIC5, CXCL13), five 2nd (PRTN3, FLJ46061/RPS28, SSX3, ABO, RGS4), and seven 3rd (ZNF3, HAPLN3, EXOC7, RFXDC2, PRRG3, MATN1, HRBL) level candidates for further evaluation by RT-PCR analysis using a larger set of untreated HRneg or Tneg breast cancers associated with long clinical follow-up (Guy's tumor set).

TABLE 3
Accession Numbers

| ProbeSetID | GeneSymbol | UniGene.ID | Entrez.Gene | RefSeq.Protein.ID | RefSeq.Transcript.ID | Group |
|---|---|---|---|---|---|---|
| 205242_at | CXCL13 | Hs.100431 | 10563 | NP_006410.1 | NM_006419 | HRneg/Tneg |
| 217628_at | CLIC5 | Hs.485489 | 53405 | NP_058625.1 | NM_016929 | HRneg/Tneg |
| 204338_s_at | RGS4 | Hs.386726 | 5999 | NP_005604.1 | NM_005613 | HRneg |
| 207341_at | PRTN3 | Hs.928 | 5657 | NP_002768.3 | NM_002777 | HRneg |
| | | | | NP_066294.1 | NM_021014 | |
| 207666_x_at | SSX3 | Hs.558445 | 10214 | NP_783642.1 | NM_175711 | Tneg |
| | FLJ46061 | Hs.322473 | 256949 | NP_001022.1 | NM_001031 | |
| 208902_s_at | RPS28 | Hs.557301 | 6234 | NP_940873.1 | NM_198471 | HRneg/Tneg |
| 216929_x_at | ABO | Hs.495420 | 28 | NP_065202.2 | NM_020469 | HRneg |
| 205523_at | HAPLN1 | Hs.2799 | 1404 | NP_001875.1 | NM_001884 | HRneg |
| 206821_x_at | HRBL | Hs.521083 | 3268 | NP_006067.2 | NM_006076 | Tneg |
| 206904_at | MATN1 | Hs.150366 | 4146 | NP_002370.1 | NM_002379 | HRneg/Tneg |
| | | | | NP_001013861.1 | NM_001013839 | |
| 212035_s_at | EXOC7 | Hs.533985 | 23265 | NP_056034.2 | NM_015219 | HRneg |
| 218430_s_at | RFXDC2 | Hs.282855 | 64864 | NP_073752.2 | NM_022841 | HRneg |
| | | | | NP_060185.1 | NM_017715 | |
| 219605_at | ZNF3 | Hs.435302 | 7551 | NP_116313.2 | NM_032924 | Tneg |
| 220433_at | PRRG3 | Hs.209253 | 79057 | NP_076987.2 | NM_024082 | HRneg |
| 201930_at | MCM6 | Hs.444118 | 4175 | NP_005906.2 | NM_005915 | HRneg |
| 202512_s_at | ATGS | Hs.486063 | 9474 | NP_004840.1 | NM_004849 | HRneg |
| 206199_at | CEACAM7 | Hs.74466 | 1087 | NP_008821.1 | NM_006890 | HRneg |
| 206378_at | SCGB2A2 | Hs.46452 | 4250 | NP_002402.1 | NM_002411 | Tneg |
| 209943_at | FBXL4 | Hs.558475 | 26235 | NP_036292.2 | NM_012160 | Tneg |
| | | | | NP_001835.2 | NM_001844 | |
| 217404_s_at | COL2A1 | Hs.408182 | 1280 | NP_149162.1 | NM_033150 | HRneg |
| 219249_s_at | FKBP10 | Hs.463035 | 60681 | NP_068758.2 | NM_021939 | HRneg |
| | | | | NP_071406.1 | NM_022123 | |
| 220316_at | NPAS3 | Hs.509113 | 64067 | NP_775182.1 | NM_173159 | Tneg |
| | | | | NP_002511.1 | NM_002520 | |
| 221923_s_at | NPM1 | Hs.557550 | 4869 | NP_954654.1 | NM_199185 | HRneg |
| 222201_s_at | CASP8AP2 | Hs.558218 | 9994 | NP_036247.1 | NM_012115 | HRneg |

### D. Example 4

The prognostic value of the HRneg and Tneg marker sets was confirmed across the combined discovery/training sets from Examples 1 and 2. This included 199 HRneg samples (135 from Example 1 and 64 from Example 2), of which 154 were Tneg (108 from Example 1 and 46 from Example 2).

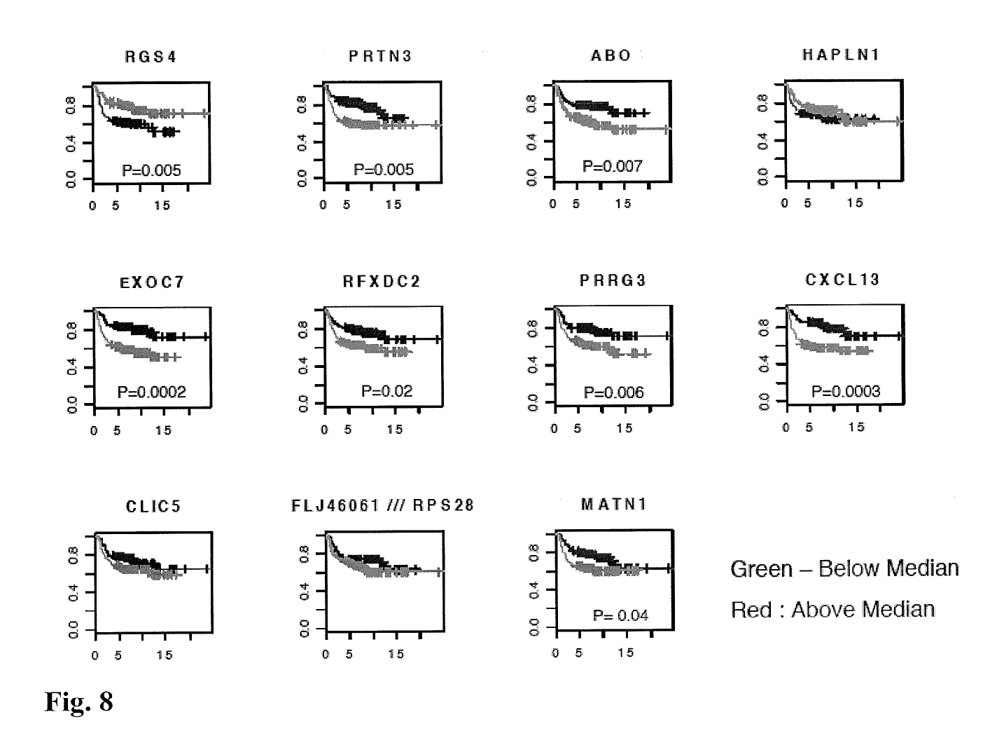

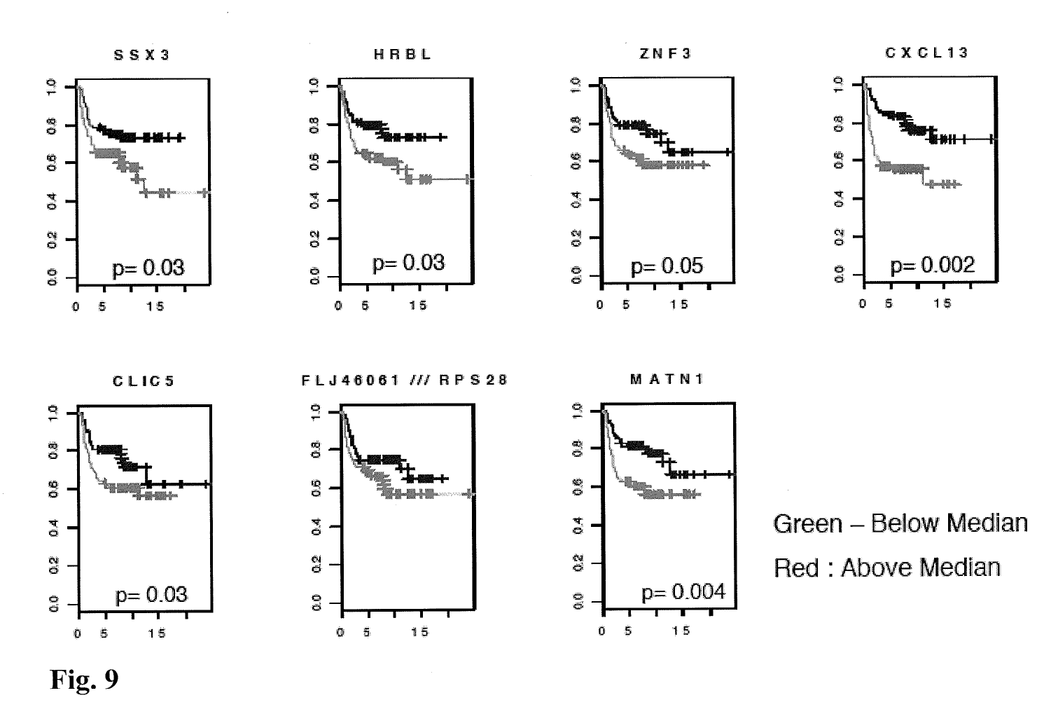

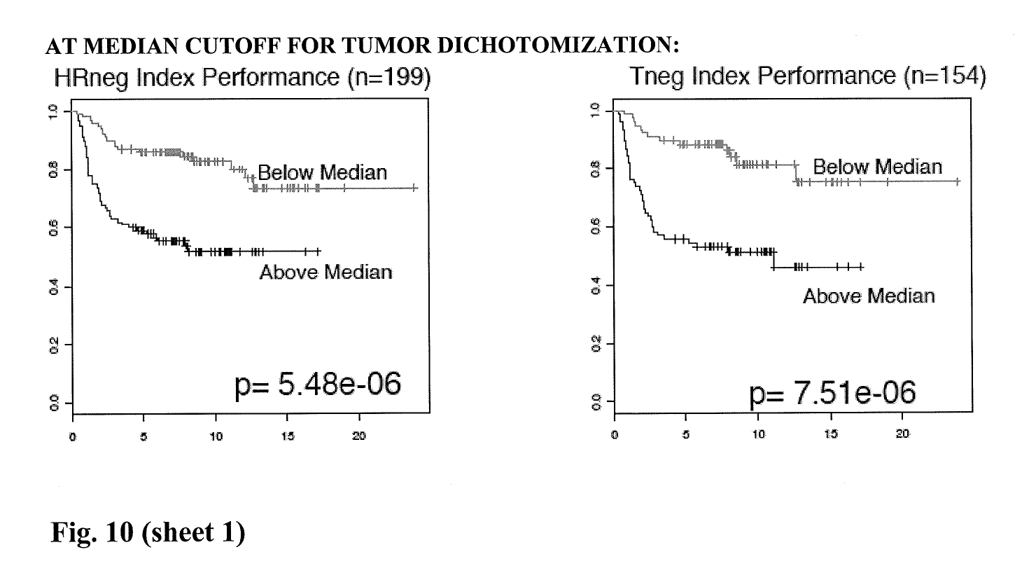

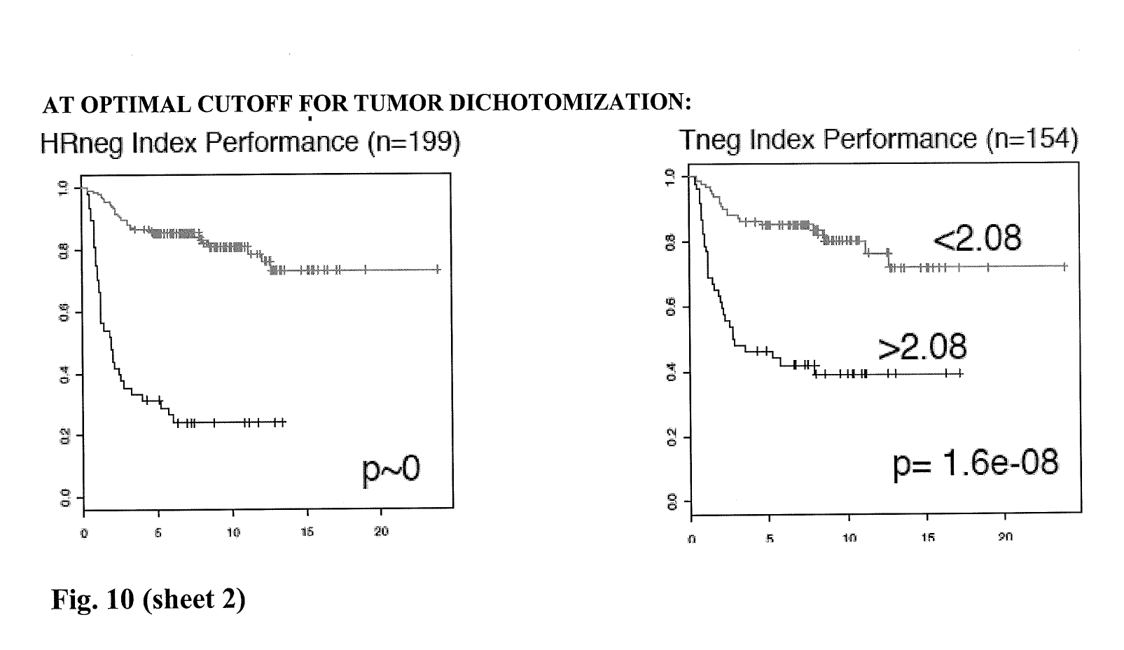

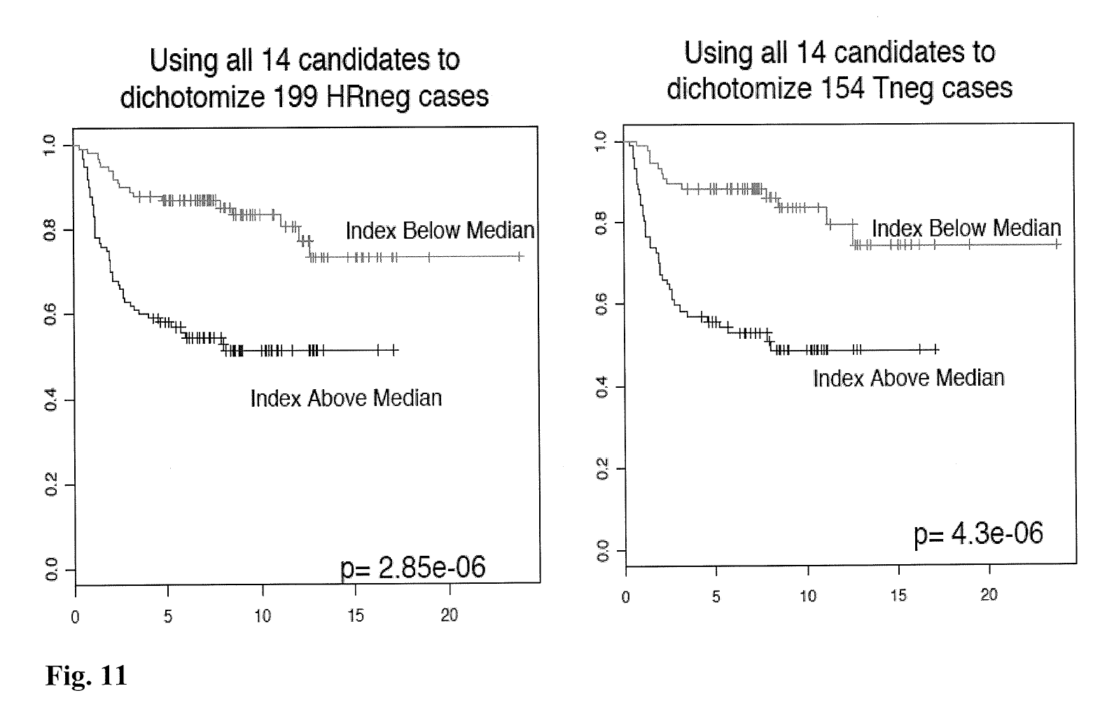

Analysis of the 18 HRneg prognostic markers resulted in selection of 11 HRneg Gene Finalists. The Kaplan-Meier analyses for the 11 individual genes, and for the summation index, are shown in FIGS. 8 and 10, respectively. Similarly, analysis of the 10 Tneg prognostic markers resulted in selection of 7 Tneg Gene Finalists. The Kaplan-Meier analyses for the 7 individual genes, and for the summation index, are shown in FIGS. 9 and 10, respectively. The HRneg and Tneg Gene Finalists selected in this combined study and their expression status are listed in Table 4. The combined summation index for all 14 Gene finalists is shown in FIG. 11 (left panel: HRneg; right panel: Tneg).

TABLE 4
Gene Finalists from combined samples

| | | Expression correlated |
|---|---|---|
| Marker Gene Symbol | Breast cancer subtype | with poor prognosis |
| RGS4 | HRneg | Increased |
| PRTN3 | HRneg | Reduced |
| ABO | HRneg | Reduced |
| HAPLN1 | HRneg | Increased |
| EXOC7 | HRneg | Reduced |
| RFXDC2 | HRneg | Reduced |
| PRRG3 | HRneg | Reduced |
| CXCL13 | HRneg/Tneg | Reduced |
| CLIC5 | HRneg/Tneg | Reduced |
| FLJ46061///RPS28 | HRneg/Tneg | Reduced |
| MATN1 | HRneg/Tneg | Reduced |
| SSX3 | Tneg | Reduced |
| HRBL | Tneg | Reduced |
| ZNF3 | Tneg | Reduced |

Univariate and multivariate COX coefficients and P values were calculated for each Gene finalist, as described in Example 1. Results are shown in Table 5. Those gene markers with greatest significance are considered top candidates for prognostic panels.

TABLE 5
COX analysis coefficients and P-values for Gene Finalists

| | Univariate COX | | Multivariate COX | |
|---|---|---|---|---|
| | analysis | | analysis | |
| Gene finalist | Coefficient | P-value | Coefficient | P-value |
| RGS4 | 0.3614 | 0.0006 | 0.3357 | 0.0001 |
| PRTN3 | −0.4075 | 0.0012 | −0.1660 | 0.2667 |
| ABO | −0.3413 | 0.0038 | −0.1549 | 0.2250 |
| HAPLN1 | 0.3439 | 0.0020 | 0.4463 | 0.0003 |
| EXOC7 | −0.4243 | 0.0002 | −0.2832 | 0.0237 |
| RFXDC2 | −0.3964 | 0.0018 | −0.3273 | 0.0121 |
| PRRG3 | −0.3984 | 0.0014 | −0.2089 | 0.1254 |
| *CXCL13 | −0.5546 | 0.0000 | −0.5201 | 0.0001 |
| *CLIC5 | −0.4417 | 0.0002 | −0.2453 | 0.0734 |
| *FLJ46061///RPS28 | −0.3694 | 0.0034 | −0.3909 | 0.0069 |
| *MATN1 | −0.3958 | 0.0014 | 0.0313 | 0.8171 |
| SSX3 | −0.3937 | 0.0050 | −03741 | 0.0102 |
| HRBL | −0.3409 | 0.0038 | 0.0877 | 0.5378 |
| ZNF3 | −03159 | 0.0051 | −0.2088 | 0.1206 |

*Gene Finalist for HRneg and Tneg breast cancer subtypes

### E. Example 5

### Comparison of Prognostic Value of 14 Gene Finalist Panel with Other Breast Cancer Gene Signatures

A new HRneg IR (immune response) gene signature includes seven genes obtained from pooled ER-negative breast cancers of all stages (Teschendorff and Caldas (2008) Breast Cancer Res. 10:R93). The gene symbols and reported P-values are listed in Table 6. Expression of the IR gene signature genes was analyzed in the 199 HRneg samples and 154 Tneg samples described in Example 4. CXCL13 expression was found to correlate strongly with that of each of the 7 IR genes, indicating that CXCL13 can be used as a proxy for the IR gene signature. CXCL13 is considered a top candidate in the present 14 gene panel, as indicated in Table 5.

TABLE 6
IR Gene Signature

| Gene symbol | P-value |
|---|---|
| C1QA | 1.41E−07 |
| HLA-F | 2.21E−13 |
| IGLC2 | 3.27E−09 |
| LY9 | 1.67E−14 |
| SPP1 | 0.002532 |
| TNFRSF17 | 3.43E−10 |
| XCL2 | 7.42E−12 |

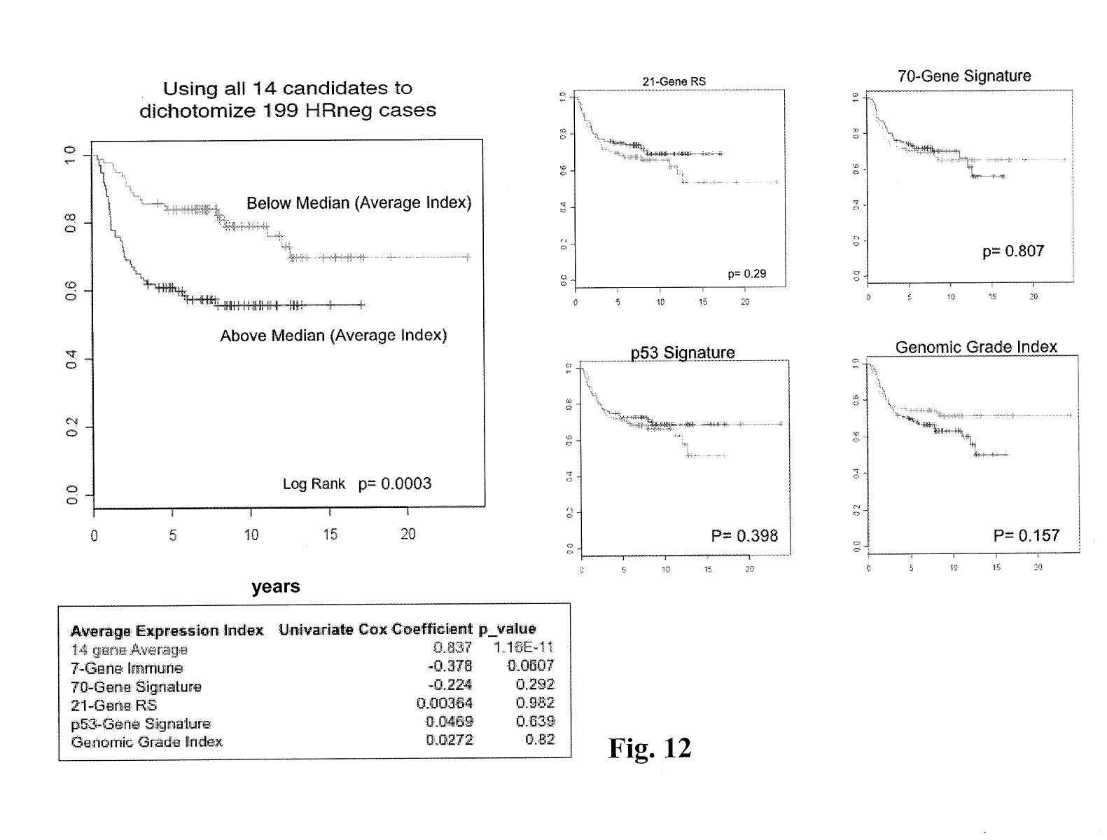

Moreover, the prognostic value of the present 14 gene signature was found to be more significant in the 199 HRneg and 154 Tneg data set than other commonly used gene signatures. A comparison of the Kaplan-Meier graphs for each signature is shown in FIG. 12.

1. A method of providing a prognosis for an individual with a Hormone Receptor negative (HRneg) or Triple negative (Tneg) breast cancer subtype, said method comprising:
(i) determining the gene expression profile of a HRneg or Tneg breast cancer subtype cell from the individual with respect to a marker set useful for the prognosis of a HRneg or Tneg breast cancer subtype; and
(ii) classifying said gene expression profile as indicating a high or low risk of metastatic relapse independent of therapy,
wherein said marker set comprises at least one gene selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN, MCM6, ATG5, COL2A1, FKBP1O, NPM1, CASPSAP2, CEAC AM7, FBLX4, NPAS3, and SCGB2A2, thereby providing a prognosis for an individual with a HRneg or Tneg breast cancer subtype.

2. The method of claim 1, wherein said marker set comprises at least one of gene selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN.

3. The method of claim 2, wherein said marker set comprises CXCL 13.

4. The method of claim 1, further comprising adjusting the therapy for the individual based on the prognosis.

5. The method of claim 1, wherein the breast cancer subtype is HRneg.

6. The method of claim 5, wherein said marker set is selected from the group consisting of HRneg S1, HRneg S2, HRneg HP, and HRTCS.

7. The method of claim 1, wherein the breast cancer subtype is Tneg.

8. The method of claim 7, wherein said marker set is selected from the group consisting of Tneg S1, Tneg S2, Tneg HP, and HRTCS.

9. The method of claim 1, wherein said expression profile is determined by RT-PCR.

10. The method of claim 1, wherein said expression profile is determined by microarray analysis.

11. A method for assigning treatment to an individual having a Hormone Receptor negative (HRneg) or Triple negative (Tneg) breast cancer subtype, said method comprising:
(i) providing a prognosis for the individual according to the method of claim 1; and
(ii) assigning a treatment to the individual based on the prognosis provided in step (i).

12. A microarray for determining the gene expression profile of a Hormone Receptor negative (HRneg) or Triple negative (Tneg) breast cancer subtype cell, said microarray comprising at least two oligonucleotide probes complimentary to genes selected from the group consisting of: CXCL13, HAPLN1, FLJ46061///RPS28, RGS4, SSX3, RFXDC2, EXOC7, CLIC5, ZNF3, PRRG3, ABO, PRTN3, HRBL, MATN, MCM6, ATG5, COL2A1, FKBP1O, NPM1, CASPSAP2, CEACAM7, FBLX4, NPAS3, and SCGB2A2.

13. The microarray of claim 12, wherein said microarray comprises oligonucleotide probes complementary to: CXCL13, HAPLN1, FLJ46061///RPS28, RGS4, SSX3, RFXDC2, EXOC7, CLIC5, ZNF3, PRRG3, ABO, PRTN3, HRBL, and MATN.

14. (canceled)

15. The method of claim 14, wherein the untreated control cell is the breast cancer cell detected in step (i) prior to contacting with the test agent.

16. The method of claim 14, wherein the untreated control cell is a breast cancer cell of the same subtype as the breast cancer cell detected in step (i).

17. The method of claim 14, wherein said expression is determined by RT-PCR.

18. The method of claim 14, wherein said expression is determined by microarray analysis.
